(19) United States
(12) Patent Application Publication
(10) Pub. No.: US 2025/0107723 A1
(43) Pub. Date: Apr. 3, 2025

(54) DERMAL SENSOR STRUCTURED TO PROVIDE CONSTANT CONTACT PRESSURE BETWEEN ELECTRODES AND A USER

(71) Applicant: Toyota Motor Engineering & Manufacturing North America, Inc., Plano, TX (US)

(72) Inventors: Frederico Marcolino Quintao Severgnini, Ann Arbor, MI (US); Parveen Singh, Dearborn, MI (US); Ercan Mehmet Dede, Ann Arbor, MI (US); Hossein Hamidi Shishavan, New Britain, CT (US); Paul Donald Schmalenberg, Ann Arbor, MI (US)

(21) Appl. No.: 18/480,203

(22) Filed: Oct. 3, 2023

(51) Int. Cl. A61B 5/0531; A61B 5/00

(52) U.S. Cl. CPC A61B 5/0531; A61B 5/681; A61B 2562/0209; A61B 2562/043; A61B 2562/164

(57) ABSTRACT

A dermal sensor includes an electrode support structure having at least one multi-material support structure first layer, at least one multi-material support structure second layer positioned opposite the first layer, and a plurality of connecting walls extending between and connecting the at least one support structure first layer and the at least one support structure second layer. The sensor also includes at least a pair of spaced-apart electrodes supported by the support structure.

### TECHNICAL FIELD

The subject matter described herein relates to sensors configured to be in contact with a skin surface of a user and, more particularly, to sensors which operate by maintaining contact pressure with the skin surface during use.

### BACKGROUND

A wearable device may incorporate a dermal sensor including electrodes designed to be in contact with a skin surface of a user, to acquire data relating to bodily functions of the user. Examples of such sensors include electrodermal activity (EDA) sensors, electrocardiogram (ECG), and electromyogram (EMG) sensors. For example, an electrodermal activity (EDA) sensor is configured to measure electrodermal activity (EDA) in a user wearing the device. The sensor may include EDA electrodes structured to physically contact a skin surface of a body part of the user. Data gathered during skin contact may be processed to provide estimates of electrodermal activity relating to the user.

To provide the most accurate available data, it is desirable that constant contact pressures be maintained between the user's skin surface and the electrodes. However, expansion and contraction of the user's body part during breathing or physical activity may cause wide variations in the contact pressures, possibly resulting in degradation of the acquired data, which may lead to inaccurate information regarding bodily functions of the user.

### SUMMARY

A dermal sensor is provided. The sensor includes an electrode support structure having at least one multi-material support structure first layer, at least one multi-material support structure second layer positioned opposite the first layer, and a plurality of connecting walls extending between and connecting the at least one support structure first layer and the at least one support structure second layer. The sensor also includes at least a pair of spaced-apart electrodes supported by the support structure.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

It will be appreciated that for simplicity and clarity of illustration, where appropriate, reference numerals may have been repeated among the different figures to indicate corresponding or analogous elements. Also, similar reference numerals appearing in different views may refer to similar elements appearing in those views. In addition, the discussion outlines numerous specific details to provide a thorough understanding of the embodiments described herein. Those of skill in the art, however, will understand that the embodiments described herein may be practiced using various combinations of these elements.

FIG. 1 is a schematic side view of a wearable device incorporating a dermal sensor in the form of an electrodermal activity (EDA), in accordance with an embodiment described herein.

FIG. 2 is a schematic side view of a portion of the dermal sensor shown in FIG. 1.

FIG. 3A is a schematic side view (through a side cross-section of the attachment structure of FIG. 1) of one example of a support structure unit cell suitable for the purposes described herein.

FIG. 3B is a schematic plan view of the unit cell shown in FIG. 3A.

FIG. 4 is a schematic partial cross-sectional view of the sensor mounted in the attachment structure.

FIG. 5 is a curve showing the relationship between displacement of the one of the first and second layers toward the other one of the first and second layers and a resulting reaction force generated by a unit cell structured in accordance with an embodiment described herein.

FIGS. 5A-5C are schematic side views of an exemplary unit cell of the support structure during various stages of deformation.

FIG. 6 is a schematic view of the dermal sensor shown in FIGS. 1-4, showing additional components and features which may be incorporated into the sensor.

### DETAILED DESCRIPTION

This disclosure relates to dermal sensors. For purposes described herein, a “dermal sensor” is a sensor designed to be in actual physical contact with a skin surface of a user, for acquiring data (relating to perspiration and/or electrodermal activity, for example) that can be processed to provide indicia of user bodily functions.

In one or more arrangements, the sensor may include an electrode support structure having at least one multi-material support structure first layer, at least one multi-material support structure second layer positioned opposite the first layer, and a plurality of connecting walls extending between and connecting the at least one support structure first layer and the at least one support structure second layer. The sensor also includes at least a pair of spaced-apart electrodes supported by the support structure. The support structure first layer may move or displace relative to the support structure second layer when an attachment structure (e.g., a wristband) incorporating the support structure is worn by a user. The support structure may be structured to provide a quasi-zero/negative stiffness response over at least a first predetermined range of displacements of one of the support structure first and second layers in a direction toward the other one of the support structure first and second layers. The support structure may also be structured to provide a quasi-zero/negative stiffness response over a second predetermined range of displacements of one of the support structure first and second layers in a direction toward the other one of support structure first and second, the second predetermined range of displacements being different from the first predetermined range of displacements. Provision of quasi-zero/negative stiffness response regions in the electrode support structure enables a constant contact pressure to be maintained between the electrodes and a skin surface of the user over the first and second predetermined range of displacements, when the attachment structure is worn by the user.

To illustrate the operating principles of the dermal sensors described herein, aspects of the invention will be described in terms of an electrodermal activity (EDA) sensor. However, it will be understood that these operating principles may apply to any dermal sensor which employs electrodes or electrical contacts in contact with a user's skin surface, and for which it is desirable to maintain a consistent level of contact pressure between the electrodes and the user's skin surface for reliable operation and provision of accurate data for processing.

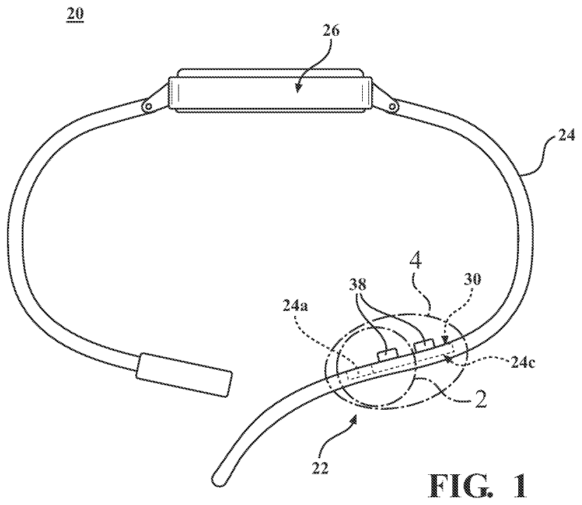

FIG. 1 is a schematic side view of a wearable device 20 incorporating a dermal sensor in the form of an electrodermal activity (EDA) sensor 22, in accordance with an embodiment described herein. As used herein, “sensor” means any device, component and/or system that can detect, and/or sense something. The EDA sensor 22 described herein can be configured to detect, and/or sense in real-time. As used herein, the term “real-time” means a level of processing responsiveness that a user or system senses as sufficiently immediate for a particular process or determination to be made, or that enables a processor(s) to keep up with some external process.

In one or more arrangements, the EDA sensor 22 may be secured to an attachment structure 24 configured to hold the EDA sensor 22 and to position and maintain the electrodes of the sensor in contact with a skin surface of a user (not shown) when the attachment structure 24 is secured to a portion of the user's body. In the embodiment shown in FIG. 1, the attachment structure 24 is in the form of a wrist band, such as a band adapted to secure a wrist watch 26 on a user's wrist. In the arrangement shown in FIG. 1, the sensor 22 is mounted in a cavity 24a formed in a portion of the band. Other arrangements are possible. In alternative arrangements, the attachment structure may be in the form of a finger ring (not shown), ankle bracelet, or any similar structure configured to wrap around a body part that is subject to expansion and contraction during user physical activity.

Generally, an attachment structure suitable for purposes described herein may be formed from a material capable of providing a relatively hard or firm surface against which an electrode support structure 30 (described in greater detail below) of the sensor 22 may rest and be supported, thereby enabling the support structure to be resiliently deformed during sensor use as described herein. The attachment structure material may be bendable (such as a flexible polymer material) or rigid (i.e., incapable of being bent by a user when used in its normal application, such as a metallic material used to form a finger ring).

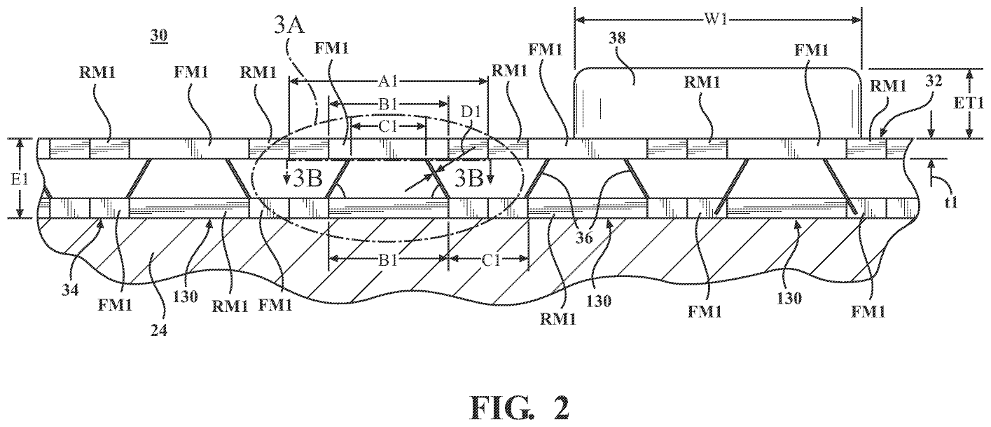

FIG. 2 is a schematic side view of a portion of the EDA sensor 22 shown in FIG. 1. Referring to FIGS. 1 and 2, the sensor 22 may include an electrode support structure (generally designated 30). In one or more arrangements, the electrode support structure 30 includes at least one multi-material support structure first layer 32 and at least one multi-material support structure second layer 34 positioned opposite the first layer 32 and extending parallel to the first layer. A plurality of connecting walls 36 may extend between and connect the at least one support structure first layer 32 and the at least one support structure second layer 34. In addition, at least a pair of spaced-apart electrodes 38 may be supported by the support structure 30. When resiliently deflected or deformed by displacement of one of the support structure first layer 32 and second layer 34 with respect to the other one of the support structure first layer 32 and second layer 34, the connecting walls 36 may generate reaction forces resisting movement of the one of the support structure first layer 32 and second layer 34 with respect to the other one of the support structure first layer 32 and second layer 34. The sum of these reaction forces provides the contact pressures between the electrodes 38 and the user's skin surface.

In one or more arrangements, the support structure first layer 32 and the support structure second layer 34 may have equal thicknesses t1. Also, each of the support structure first layer 32 and the support structure second layer 34 may be a multi-material layer (i.e., a layer formed as a single, unitary piece from more than one material). Referring to FIG. 2, for example, in one or more arrangements, each of electrode support structure multi-material first and second layers 32, 34 may comprise alternating sections of a relatively rigid material RM1 and a relatively flexible material FM1.

For purposes described herein, a “flexible material” as used in the support structure may be a material usable to fabricate connecting walls 36 so that the walls will resiliently deform under the anticipated loading resulting from displacement of one of the support structure first layer 32 and second layer 34 with respect to the other one of the support structure first layer 32 and second layer 34. “Resiliently deform” refers to a tendency of an element to return to its undeformed or undeflected state upon removal of an applied loading as described herein (i.e., the element will elastically deform rather than plastically or permanently deforming due to the anticipated loading). Flexible materials usable for the purposes described herein may be configurable into structures that can provide the force-deflection responses (including quasi-zero/negative stiffness region(s)) described herein and may also be amenable to 3D printing operations. Examples of flexible materials usable for the purposes described herein include various rubber compounds. In addition, the connecting walls 36 may be structured to exhibit non-linearly elastic deformation when subjected to loads caused by expansion of a user's body part. This structure of the connecting walls 36 may provide a quasi-zero/negative stiffness response capability to the support structure, as described herein.

Other types of materials adaptable for the flexible material applications described herein include hydrogels, reconfigurable architected materials, liquid crystal elastomers, and vitrimers. Vitrimers are derived from thermosetting plastics and can be processed to fabricate specific materials with desirable properties. Generally, Vitrimers exhibit both flexibility and spring back properties. Vitrimers can also be processed using additive manufacturing methods, such as 3-D printing.

For purposes described herein, a “rigid material” used in the support structure may be a material usable to fabricate elements of the electrode support structure 30 so that they will not deform (or may deform to a degree insignificant to operation of the support structure), responsive to the anticipated loading resulting from displacement of one of the support structure first layer 32 and second layer 34 with respect to the other one of the support structure first layer 32 and second layer 34. Examples of relatively rigid materials suitable for the purposes described herein include polymers such as acrylics or various grades of ABS (Acrylonitrile Butadiene Styrene).

The sections formed from relatively rigid material RM1 and the sections formed from relatively flexible material FM1 may have equal thicknesses t1. As seen in FIG. 2, the connecting walls 36 may be formed from the flexible material and may connect sections of the support structure second layer 34 formed from the flexible material with a section of the support structure first layer 32 formed from the flexible material FM1.

In one or more arrangements, the electrode support structure second layer 34 may extend parallel to the support structure first layer 32. Each of the connecting walls 36 extending between the layers 32, 34 may form an associated acute angle θ with each of the first layer 32 and the second layer 34 (i.e., the layers 32, 34 and each connecting wall 36 may form a set of alternate interior angles θ with respect to the parallel first and second layers 32, 34). In particular arrangements, the angle θ may be in the range 60°+25°.

Flexible material sections of the support structure first and second layers 32, 34 may be structured to permit a degree of bending transverse to a length of the section. This may facilitate a degree of bending and/or buckling of the overall electrode support structure 30 to conform to a contour of the user's skin surface when the attachment structure 24 is worn by the user.

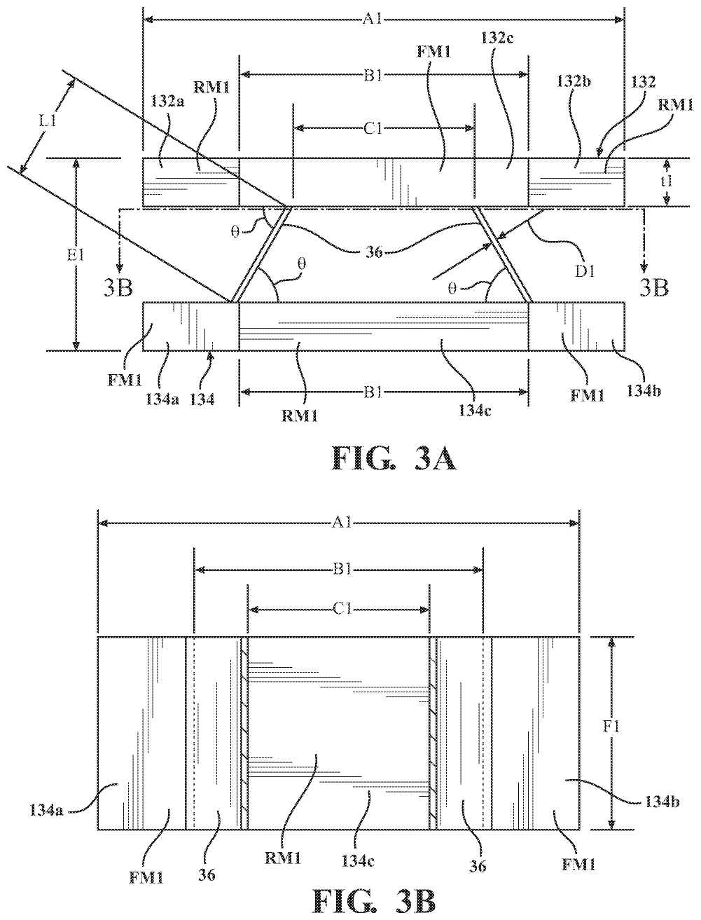

Referring to FIGS. 2, 3A and 3B, in particular arrangements, the electrode support structure 30 is formed from a plurality of support structure unit cells (generally designated 130) formed as a single piece or mechanically connected end-to-end in “bracelet” fashion using suitably-designed complementarily interconnecting microstructures formed at ends of the individual cells. FIG. 3A is a schematic side view (taken through a side cross-section of the attachment structure 24 of FIG. 1) of one example of a support structure unit cell 130 suitable for the purposes described herein. FIG. 3B is a schematic plan view of the unit cell shown in FIG. 3A. Alternative structures may also be used. An embodiment of the unit cell 130 may incorporate the features described with regard to FIG. 2 in an arrangement that facilitates formation or connection of a plurality of the unit cells in an end-to-end configuration to form the overall electrode support structure 30. FIG. 2 also shows four unit cells 130 formed contiguously as a single piece so as to be attached end-to-end. However, any desired number of unit cells 130 may be formed contiguously or as “links” in “bracelet” fashion to provide the overall electrode support structure 30.

Referring to FIGS. 3A and 3B, in one or more arrangements, each unit cell 130 may have the same structure and may include a multi-material unit cell first layer 132 and a multi-material unit cell second layer 134 positioned opposite the unit cell first layer 132 and extending parallel to the unit cell first layer. A plurality of connecting walls 36 may extend between and connect the unit cell first layer 132 and the unit cell second layer 134. In the arrangement shown in FIGS. 3A and 3B, a pair of connecting walls 36 connects the unit cell first and second layers 132, 134. In addition, each of the unit cell first and second layers 132, 134 may be formed from alternating the relatively rigid material RM1 sections and relatively flexible material FM1 sections, as previously described with respect to the overall electrode support structure 30.

For example, in unit cell 130, first and second end portions 132a, 132b, respectively, of unit cell first layer 132 each comprise rigid sections formed from a relatively rigid material RM1 as previously described, while an intermediate section 132c of the unit cell first layer 132 extending between the first and second end portions 132a, 132b comprises a flexible section formed from a relatively flexible material FM1 as previously described. Also, first and second end portions 134a, 134b of unit cell second layer 134 each comprise flexible sections formed from the relatively flexible material FM1, while an intermediate section 134c of the unit cell second layer 134 extending between the first and second end portions 134a, 134b is a rigid section formed from the relatively rigid material RM1. In addition, as shown in FIG. 3A, the connecting walls 36 may have equal lengths L1, may be formed from the relatively flexible material, and may extend between (and connect) the relatively flexible sections of the unit cell first and second layers 132, 134. Thus, walls 36 connect flexible sections 132c of unit cell first layer 132 to the first and second flexible end portions 134a and 134b of the unit cell second layer 134.

Referring to FIGS. 2 and 3A-3B, in the drawing figures and the description:
A1=an overall length of a single support structure unit cell 130;
B1=a length of a flexible intermediate section 132c of a unit cell first layer/electrode support structure first layer (and also a length of a rigid section 134c of a unit cell second layer 134 positioned opposite the flexible section 132c);
C1=a minimum spacing between adjacent and opposed connecting walls 36 of a unit cell;
D1=thickness of the connecting walls 36;
E1=an overall thickness of a unit cell/the electrode support structure;
F1=an overall width of a unit cell/the electrode support structure;
L1=a width of a connecting wall extending between the first and second layers of a unit cell/the electrode support structure;
θ=an angle formed between the connecting wall and the parallel first and second layers of a unit cell/electrode support structure;
t1=a thickness of each of the first and second layers of a unit cell/electrode support structure
   Values of various parameters of the unit cell 130 may be adjusted to provide a desired or customized force-deflection response of the unit cell. For example, the length B1 of the rigid section 134c of second layer 134 may be adjusted to accommodate changes in any or all of parameters C1, L1, and/or θ. Also, angle θ, connecting wall thickness D1, connecting wall length L1 and/or overall thickness E1 may be modified to adjust to location(s) and other characteristics of one or more quasi-zero/negative stiffness region(s) on the force-deflection curve.

In one or more arrangements, values of the unit cell parameters may be controlled to provide a single unit cell design structured so that a plurality of the unit cells can be “chained/linked” or formed as a single piece end-to-end to provide an electrode support structure having a desired force-deflection response (including one or more quasi-zero/negative stiffness region(s)), and also to promote uniformity of the support structure and the response characteristics of the supports structure. Referring to FIG. 3A, for example, dimensions of unit cell sections formed from flexible material FM1 at ends 134a and 134b of unit cell second layer 134 may be controlled so that they are equal and also so that a minimum spacing between the connecting walls 36 may be equal to the value of dimension C1 when two unit cells are formed end-to-end.

Any desired number of unit cells 130 may be formed into an electrode support structure 30 as shown in FIG. 2 depending on the sizes of the electrodes 38, the desired coverage area of the electrode support structure over the user's skin surface, and other pertinent factors.

In one or more arrangements, sections of an individual unit cell formed from different materials (and also contiguous individual unit cells in a plurality of unit cells formed or attached end-to-end) may be integrated into the support structure in the manner shown in FIGS. 2 and 3A-3B by the use of additive manufacturing techniques (e.g., three-dimensional or 3D printing). For example, a suitable multi-material three-dimensional (3-D) printing process may be used to fabricate the unit cells 130 and the electrode support structure 30. One example of a 3D printer which is configurable to produce unit cells, electrode support structures formed from the unit cells, and electrodes as described herein is the Objet500 Connex3™ 3D printer available from Stratasys Ltd. Of Eden Prairie, Minnesota. Other equipment or printers may also be suitable for producing embodiments of the unit cells and electrode support structures.

Any embodiment of the EDA sensor 22 may include a pair of electrically-isolated, spaced-apart electrodes 38. As used herein, the term “electrically-isolated” means that the electrodes 38 are mounted on the electrode support structure 30 so as to be physically spaced apart from each other by an electrical insulator, such as an air space and/or a section of the electrode support structure formed from an insulating material, for example.

Regarding the spacing between the electrodes 38, increasing the electrode spacing distance may reduce standard deviations between measurements, thereby providing more repeatable results. However, a relatively smaller inter-electrode space leads to a stronger electric field within a smaller skin volume. These competing effects may be balanced according to the requirements of a given application, and in consideration of the surface area on the attachment structure 24 available for incorporating the EDA sensor 22 thereon. The electrodes 38 may have minimal thicknesses ET1 (FIG. 2) needed to perform the electrode functions described herein.

In one or more arrangements, the electrodes 38 may be formed from associated thin layers of gold, a gold alloy, silver, a silver alloy, or any other suitable electrode material(s). However, the electrodes 38 may include one or more conductive polymers, semiconductors, or other conductive materials to minimize electrical resistance and/or electrical polarization. It will be appreciated that any suitable conductive material or materials may be utilized for the electrodes. In one or more particular arrangements, the electrodes 38 may be formed from an electrically conductive, elastically deformable material structured to conform to any curvature of the support structure resulting from wearing of the attachment structure by a user. For example, materials suitable for the electrodes may include various known textile fabrics coated with modified poly (3,4-ethylenedioxythiophene):poly (styrenesulfonate) (PEDOT:PSS) in order to provide reliable and washable textile electrodes that may be incorporated into items of clothing, such as underwear. Such electrodes are known and are described in a paper entitled “Washable and Reliable Textile Electrodes Embedded into Underwear Fabric for Electrocardiogra (ECG) Monitoring” by Amale Ankhili et al., the content of which is incorporated herein by reference in its entirety.

The electrodes 38 may be supported by the support structure 30 in a manner such that, when the support structure is mounted on a suitable attachment structure 24, the electrodes will simultaneously be in contact with a skin surface of a user when the user is “wearing” the attachment structure 24. The electrodes 38 may be structured to contact an exposed skin surface of a user when the support structure is secured to a suitable attachment structure and the attachment structure is secured to a portion of the user's body. Each of electrodes 38 may have a generally cylindrical shape, a cubic shape, a flat shape, or another shape. In the embodiments described herein, each electrode has a cylindrical shape with a diameter W1 supported by the electrode support structure first layer 32.

In one or more arrangements, one or more of the electrodes 38 may be formed integrally with the electrode support structure (i.e., simultaneously with formation of the support structure and/or using the same process used to form the support structure). For example, in some arrangements, one or more layers of an electrically conductive electrode material may be deposited onto a layer of a different material (e.g., a polymer material forming a portion of the support structure) using the same additive manufacturing process used to form the support structure or a different additive manufacturing process, thereby forming a conductive “patch” on the portion of the support structure configured to reside adjacent the user's skin. In other arrangements, the electrodes 38 may be formed separately from the electrode support structure 30, then attached to the electrode support structure 30 using any suitable method, such as adhesive attachment, mechanical fasteners, etc.

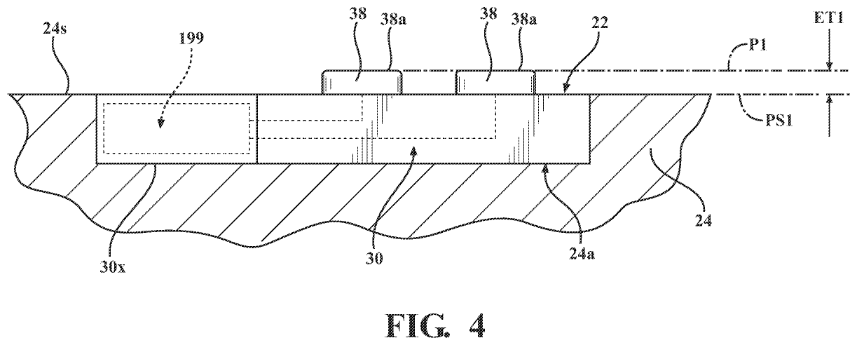

FIG. 4 is a schematic partial cross-sectional view of the sensor 22 mounted in the attachment structure 24. Referring to FIG. 4, the electrodes 38 may be mounted to the electrode support structure 30 so as to extend past a plane PS1 defined by an innermost surface 24s of the attachment structure 24 (i.e., a surface of the attachment structure facing toward the user's skin surface). The electrodes 38 may have inwardly-facing surfaces 38a structured to contact a skin surface of a user when the user is wearing a wearable device (such as watch 26) incorporating the attachment structure 24. In particular arrangements, the inwardly-facing surfaces 38a are structured to be coplanar along a flat plane P1. This arrangement helps ensure that both of electrodes 38 simultaneously contact a skin surface of the user when the user is wearing the wearable device 26. In more particular arrangements, the plane P1 may be structured to be spaced apart from, and parallel to, plane PS1. This spacing arrangement may permit the electrodes 38 to extend deeper into the skin surface for enhanced contact between the electrodes and the skin surface when the user is wearing the wearable device 26. In particular arrangements, the spacing between the planes P1 and PS1 may be in the range 0-0.040 inches.

The size of an electrode can determine the effective electrode-skin contact area, the signal-to-noise ratio of the generated EDA signal, and the sensitivity of the EDA sensor 22. To enhance these properties, it is generally beneficial to have relatively larger electrodes. However, the sizes of the electrodes 38 may be constrained by the surface area available on the electrode support structure 30. The electrodes 38 should not be too small because higher current densities resulting from small electrode-skin contact areas may increase signal generation errors due to factors such as counter EMF. In one or more arrangements described herein, and for a wearable device in the form of a finger ring, the two electrodes 38 may be structured so as to have inwardly-facing surfaces 38a of equal areas, with the sum of the areas of the inwardly-facing surfaces 38a being equal to about 0.15 centimeter².

To provide accurate EDA data, it is desirable that contact pressures between the user's skin surface and the electrodes 36 be maintained at as constant a level as possible, whether the user is at rest or physically active. The contact pressures between the electrodes and the user's skin may be applied by the electrode support structure in response to displacement of the support structure first layer 32 toward the second layer 34 during wearing of the wearable device 26. In addition, expansion of the user's body part due to muscle flexure, physiological response to exercise, hydration and/or general physical movement may increase the compression of the support structure 30 and displacement of the support structure first layer 32, and contraction of the user's body part may relieve the compression of the support structure 30, causing a relative expansion of the support structure and a return of the support structure to the initial (i.e., “at rest”) displacement.

In one or more particular embodiments, the attachment structure may be omitted. In such embodiments, the outer multi-material band formed by the combined, contiguous (or connected) second layers of the support structure may function as the attachment structure responsive to tension generated in the band during expansion of the user's body part, thereby providing the necessary reaction force on one side of the support structure to compress the unit cells.

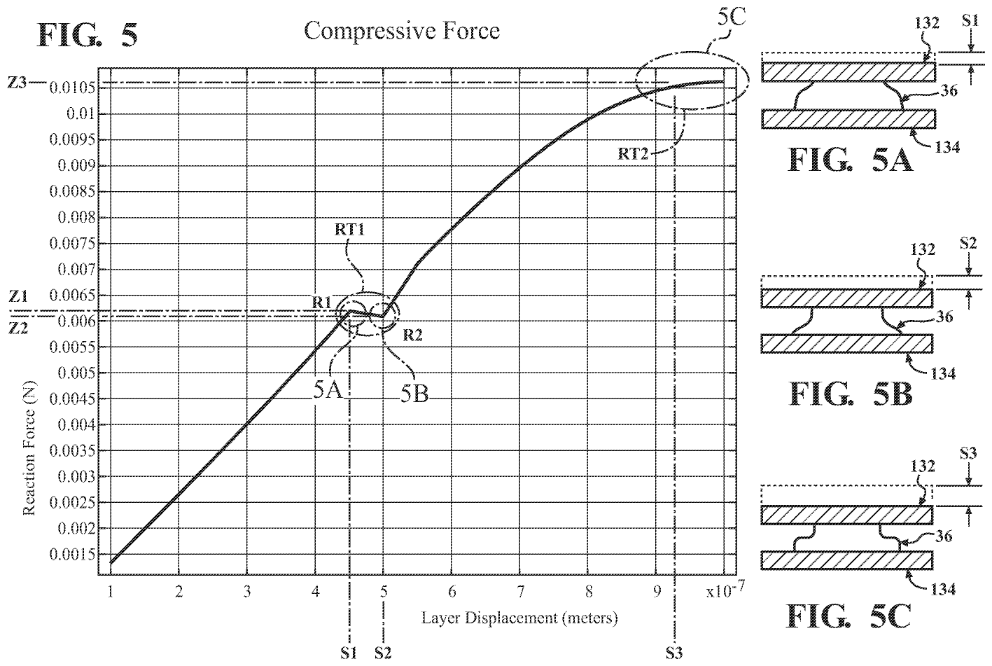

FIG. 5 is a curve showing the relationship between displacement of the one of the first and second layers toward the other one of the first and second layers and a resulting reaction force generated by a unit cell structured in accordance with an embodiment described herein. Referring to FIG. 5, it has been found that embodiments of the electrode support structure described herein may be structured to provide a “quasi-zero/negative stiffness” responsive to displacement of one of the support structure first and second layers 32, 34 in a direction toward the other one of support structure first and second layers 32, 34, over at least a first a predetermined range of displacements. FIGS. 5-5C also illustrate the nonlinearly elastic deformability of the connecting walls 36. For example, in one or more arrangements, in a support structure fabricated from multiple identically configured unit cells attached end-to-end as shown in FIGS. 2 and 3A-3B, the unit cell design may be structured such that its force-deflection curve exhibits at least one quasi-zero/negative stiffness region responsive to displacement of one of the unit cell first and second layers 132, 134 in a direction toward the other one of unit cell first and second layers 132, 134 (e.g., when the unit cell first layer 132 is moved in a direction toward the unit cell second layer 134). FIGS. 5-5C as described herein also illustrate how the connecting walls 36 may progressively buckle under the applied loading described herein.

One characteristic of the curve shown in FIG. 5 is at least a first region (generally designated RT1) in which a slope of the curve may be zero, near zero, or negative for a displacement within a specified range of displacements. The range of displacements over which this behavior occurs defines a “quasi-zero/negative” stiffness region of the force-deflection curve. In this quasi-zero/negative stiffness region, the electrode support structure (and/or one or more unit cells forming the support structure) may exert constant or near-constant reaction forces responsive to non-linear elastic deformations and/or buckling of the connecting walls within the range of displacements when a unit cell (such as unit cell 130 of FIGS. 3A-3B) is compressed so that unit cell first layer 132 moves toward unit cell second layer 134. The range of displacements over which this behavior occurs defines a “quasi-zero/negative” stiffness region of the force-deflection curve. In this quasi-zero/negative stiffness region, the unit cell may exert constant or near-constant reaction forces over the range of displacements when the unit cell is compressed.

The quasi-zero/negative region RT1 shown in FIG. 5 may extend over a range of relatively lower deflections of the unit cell first layer 132. Thus, the unit cell 130 may be structured to exert a correspondingly relatively lower constant reaction force responsive to a displacement of the unit cell first layer 132 within this range. While this reaction force may provide a relatively lower constant contact pressure between the electrodes 38 and the user's skin surface, the location of the quasi-zero/negative region along the force-displacement curve may be tailored so that the contact pressure is still sufficient for the purposes described herein. Thus, an electrode support structure formed from a plurality of such unit cells may provide reliable EDA readings for a stationary user or even a user engaged in a relatively low level of physical activity.

FIG. 5A is a schematic side view of a representative unit cell 130 illustrating resilient deformation in the form of buckling of the connecting walls 36 responsive to displacement of the unit cell first layer 132 a first amount S1 toward unit cell second layer 134 necessary to reach a location R1 on the curve defining a beginning portion of the quasi-zero/negative region RT1. Displacing the unit cell first layer 132 the first amount S1 (by expansion of a user's body part, for example) may cause the connecting walls 36 to generate a reaction force Z1 on the user's body part.

FIG. 5B is a schematic side view of the representative unit cell 130 illustrating resilient buckling of the connecting walls 36 responsive to displacement of the unit cell first layer 132 a second amount S2 toward unit cell second layer 134 necessary to reach a location R2 on the curve defining an end portion of the quasi-zero/negative region RT1. Displacing the unit cell first layer 132 the second amount S2 (by further expansion of the user's body part, for example) may cause the connecting walls 36 to generate a reaction force Z2 on the user's body part. As seen from FIG. 5, because the first layer 132 has been further displaced within the first quasi-zero/negative response region RT1 when the location R2 is reached, the reactive force Z2 generated by the further displacement S2 may (in some cases) be the same or less than the reactive force Z1 produced by displacement of the first layer 132 by S1.

Referring again to FIG. 5, it has also been found that particular arrangements of the electrode support structure 30 may be fabricated so as exhibit multiple “quasi-zero/negative” stiffness regions along a force-deflection curve of the unit cell or an electrode support structure formed from such unit cells. Thus, an electrode support structure may also be structured to provide another quasi-zero/negative stiffness response region (generally designated RT2) over a second predetermined and customizable range of displacements of one of the support structure first and second layers 32, 34 in a direction toward the other one of support structure first and second layers 32, 34, with the support structure being configured so that the second predetermined range of displacements is different from the first predetermined range of displacements.

For example, the quasi-zero/negative region RT2 shown in FIG. 5 may extend over a range of relatively greater displacements of the unit cell first layer 132 with respect to the unit cell second layer 134. Thus, the unit cell 130 may be structured to exert a correspondingly relatively higher constant reaction force responsive to a displacement within this range. Consequently, an electrode support structure formed from a plurality of such unit cells may also provide reliable EDA readings for a user engaged in a relatively higher or more intense level of physical activity (e.g., from exercise or other physical exertions) causing a greater expansion of the user's body part. FIG. 5C is a schematic side view of the representative unit cell 130 illustrating resilient deformation of the connecting walls 36 when the unit cell first layer 132 has been displaced an amount S3 toward unit cell second layer 134 necessary to reach a location on the curve defining the start of the quasi-zero/negative region RT2. Displacing the unit cell first layer 132 the second amount S3 (by further expansion of the user's body part, for example) may cause the connecting walls 36 to generate a reaction force Z3 on the user's body part.

During operation of the electrode support structure 30 when secured to an attachment structure 24 worn by a user, forces generated by contact with the user's body part are transmitted through the electrodes 38 to the electrode support structure 30, causing the support structure first layer 32 to deflect in a direction toward the support structure second layer 34, thereby compressing the connecting walls 36. With the multi-material second layer 34 of the support structure 30 bearing against the relatively unyielding surface of the attachment structure 24 material, wearing of the attachment structure 24 on a body part of the user with the electrodes 38 pressed against the user's skin surface may produce a contact force which locally displaces the support structure first layer 32 in a direction toward the support structure second layer 34, thereby compressing the electrode support structure. Distributed reaction forces may be generated by the resulting deformation of the connecting walls 36 supporting the portions of the support structure first layer 32 acted on by the electrodes 38 in contact with the user's skin surface. The electrodes 38 may be spaced apart close enough to each other along the support structure first layer 32 so that localized expansion of the portion of the user's body part will produce substantially equal displacements of the portions the first layer 32 supporting the electrodes 38. The support structure 30 may be designed and fabricated so that these displacements will be within the specified range of displacements needed to produce a quasi-zero/negative stiffness response in the connecting walls 36.

Based on electrode areas to be in contact with the user's skin surface, and the contact pressures required (or desired) for generation of accurate electrodermal activity measurements, forces needed to provide sufficient contact pressure with the user's skin surface may be determined. Ranges of expansion of the user's body part under different conditions of physical activity may be estimated. The electrode support structure may then be designed to tailor QZS response region(s) to exert constant or substantially constant forces on the electrodes, so as to generate associated constant or substantially constant contact pressures over the estimated range of body part expansion.

The localized displacement of the support structure first layer 32 and the associated reaction forces produced by the aggregated deformation of the supporting connecting walls 36 may produce composite reaction forces that generate contact pressures between the electrodes 38 and the user's skin surface. Also, because the localized displacement produces reaction forces in the quasi-zero/negative stiffness zones of the connecting walls 36, slight variations in the localized first layer displacement may not appreciably affect the generated reaction forces. As a result, a constant or substantially constant reaction forces may be generated by the deflected electrode support structure responsive to any displacement of the first layer 32 within the specified range of displacements. These reaction forces are transmitted through the electrodes 38 as constant or substantially constant contact pressures between the electrodes and the user's skin surface. In this manner, a constant contact pressure (within a relatively small tolerance range) may be maintained during wearing of the EDA sensor by a user engaged in a constant level of physical activity.

It has been found possible to provide a desired quasi-zero/negative stiffness region in a given embodiment of the electrode support structure by tailoring the values of certain design parameters to the meet the electrode contact pressure requirements of a particular application. Key parameters may include an anticipated range of displacements of the support structure first layer 32 relative to the support structure second layer 34; material properties of the relatively flexible connecting wall material; the angle θ of the connecting walls 36 with respect to the support structure first and second layers 32, 34; the length L1 of the connecting walls 36; the thickness D1 of the connecting walls 36; the minimum spacing C1 between the connecting walls; bearing areas of the electrodes 38 on the support structure first layer 32 (which affects the number of connecting walls supporting the electrodes), and other parameters. The anticipated range of displacements may be based, for example, on data relating to anticipated expansion of a user's body part (such as the wrist) during a specific type of physical activity (e.g., no activity/light activity vs. relatively intense activity) performed by the user. More intense activity (such as strenuous and/or prolonged physical exercise) may produce a greater expansion of the body part, thereby producing a correspondingly greater displacement of the support structure first layer in a direction toward the support structure first layer. As part of a design optimization process, for example, the support structure described herein may be modeled and one or more of the parameters listed above may be iteratively adjusted, analytically and/or through experimentation, to determine an associated quasi-zero/negative stiffness region that generates a constant reaction force (within a certain tolerance range) within a predetermined range of anticipated displacements of the support structure first layer in a direction toward the support structure second layer.

Alternatively, an estimated range of displacements for the first layer of the support structure can be based on anticipated pressures to be applied to the first layer. This method of estimating displacements may be particularly relevant when the support structure is incorporated into a finger ring, for example, where pressure variations arise from expansion and contraction of the user's finger as the user opens and closes his/her hand.

### Example

Referring to FIG. 5, in one non-limiting example, a representative unit cell 130 was prepared including the following parameter values:
F1=1.5 millimeters
E1=2 millimeters
θ=60°
D1=50-300 μm
First and second layer thickness t1=500 μm
A1=5 millimeters

A force was then applied to the unit cell first layer 132 to produce a displacement of the first layer simulating expansion of a user's body part (e.g., a user's wrist), resulting in the force-deflection curve shown in FIG. 5. As stated previously, FIGS. 5A-5C show schematic side views of the sample unit cell 130 at various stages of the progressive deformation of the connecting walls 36 corresponding to associated locations on the force-deflection curve. It may be seen from the curve that the resiliently deformable connecting walls 36 act as non-linear spring members during displacement of the unit cell first layer 132 with respect to the unit cell second layer 134.

As seen from FIG. 5, a first quasi-zero/negative stiffness region RT1 may be created providing a reaction force of about 0.006 Newton over a range of first layer displacements of about 4.5×10⁻⁷ meter to 5.0×10⁻⁷ meter. More specifically, the curve shows the first quasi-zero/negative stiffness region RT1 bounded by a location R1 corresponding to a reaction force of about 0.0062 Newton at a displacement of 4.5×10⁻⁷ meter and a location R2 corresponding to a reaction force of about 0.0061 Newton at a displacement of 5×10⁻⁷ meter. In addition, a second quasi-zero/negative stiffness region RT2 is created providing a reaction force of about 0.0105 Newton over a range of first layer displacements of about 9×10⁻⁷ meter to 10.0×10⁻⁷ meter. Thus, design described in the example may produce a constant or substantially constant reaction force responsive to particular ranges of displacement. Through design optimization, the quasi-zero/negative stiffness regions may be tailored so that the ranges of displacement correspond to the projected expansion of a user's body part during various types of physical activity states. The displacement ranges achieved may also allow for some slight variations in the body part expansion of a particular user and for some variations in the amount that body parts of different users will expand when engaged in similar levels of physical activity. In addition, when the user's body part is moving or experiencing low-amplitude vibrations, the connecting walls 36 may act as vibration absorbers while maintaining a desired substantially constant reaction force (i.e., a reaction force within a certain narrow range of forces).

It has been found through analysis and experimentation that, in certain arrangements, values of the connecting wall thickness D1 and the connecting wall angle θ may have a relatively greater influence that the other unit cell parameters on the characteristics of the quasi-zero/negative stiffness region(s) and the location of the region(s) on the force-deflection curve. Thus, in certain cases, it may be feasible to adjust or “tune” the design of the electrode support structure to achieve targeted quasi-zero/negative stiffness response(s) by varying values of one or more of these parameters.

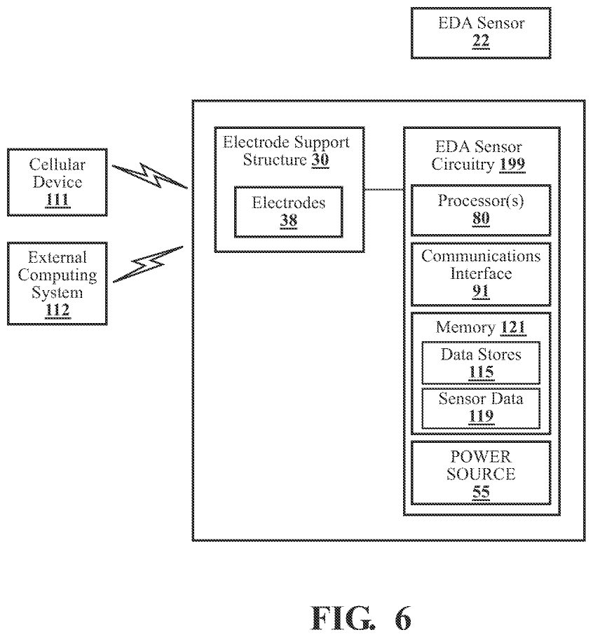

FIG. 6 is a schematic view of the EDA sensor showing additional components and features which may be incorporated into the sensor. Referring to FIG. 6, in one or more arrangements, EDA circuitry 199 may be communicatively coupled to the electrodes using wires, conductive traces, or any other suitable method. The EDA circuitry 199 may be incorporated into the sensor 22 as part of the sensor or the EDA circuitry 199 may be separate from the sensor and configured to communicatively couple to the electrodes 38 when mounted on an associated attachment structure 24 as described herein. The EDA circuitry 199 may be configured to generate an EDA signal using information acquired through physical contact between the electrodes 38 and a skin surface of a user. The EDA signal may be a signal indicative of electrodermal activity associated with the user wearing the wearable device 26. For example, the EDA signal may be indicative of a measurement of conductance or resistance associated with one or more dermal layers of the user's skin. The user's perspiration can provide electrical contact between the electrodes 38 and the skin surface of the user, thereby facilitating ionic flow between the electrodes 38 and the skin surface. The EDA circuitry 199 and electrodes 38 can induce a current through one or more dermal layers of a user's skin. The current transmitted between the electrodes through the user's skin can be measured and correlated in a known manner to determine conductance, resistance, or another measure indicative of sympathetic nervous system activity. In one or more arrangements, the EDA circuitry 199 may cause EDA signals to be generated automatically and periodically whenever the electrodes 38 are in physical contact with a user's skin surface.

EDA circuitry 199 can include various components such as amplifiers, filters, charging circuits, sense nodes, and other elements configured to sense one or more electrical characteristics of a user responsive to current transmission through the electrodes. EDA circuitry 199 can be implemented as voltage sensing circuitry, current sensing circuitry, capacitive sensing circuitry, resistive sensing circuitry, etc.

Referring to FIG. 4, in one or more arrangements, at least a portion of the EDA circuitry 199 may be incorporated into an embodiment of the electrode support structure 30. For example, FIG. 4 shows EDA circuitry 199 positioned and secured in a cavity or receptacle 30x formed in the support structure 30. The cavity 30x may be formed during fabrication of the support structure 30 using the same additive manufacturing process(es) used to form the support structure. The circuitry 199 may then be positioned in the cavity 30x and operably connected to the electrodes 38 to provide a self-contained, modular EDA sensor that can be secured to the attachment structure.

Referring to FIG. 4, in one or more alternative arrangements, one or more elements of the EDA circuitry 199 may be positioned in the attachment structure cavity 24a adjacent or spaced apart from the electrode support structure 30 and electrodes 38. The one or more elements of the EDA circuitry may then be operably connected to the electrodes 38.

In one or more arrangements, the EDA circuitry 199 may include a processor 80, a memory 121 and/or other elements usable for performing EDA sensing and other computational and/or decision-making functions of the sensor. The EDA sensor circuitry 199 may cause EDA signals to be generated automatically and periodically whenever the electrodes 38 are in physical contact with a user's skin surface. In some particular arrangements, the EDA circuitry 199 may be configured to cause EDA signals to be generated at specific times or at regular time intervals based on specified predetermined conditions.

Referring again to FIG. 6, in one or more arrangements, the sensor circuitry 199 may also include a power source 55 (such as a battery) operably connected to other elements of the wearable device 26 and configured for powering the various operations performed by the processor(s) 80, the EDA sensor circuitry 199, and other active elements of the wearable device 26. For example, power source 55 may be coupled to EDA sensor circuitry 199 to provide power to the sensing circuitry to enable transmission of an electric current through electrodes 38. The power source 55 may be removable or embedded within the wearable device 26.

Referring to FIG. 1, the sensor circuitry 199 can include one or more processor(s) 80. The sensor circuitry 199 can also include a memory 121 incorporating one or more data stores 115 for storing one or more types of data. The data store(s) 115 can include volatile and/or non-volatile memory. Examples of suitable data store(s) 115 include RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory) or any other suitable storage medium, or any combination thereof. The data store(s) 115 can be a component of the processor(s) 80, or the data store(s) 115 can be operably connected to the processor(s) 80 for use thereby. The term “operably connected,” as used throughout this description, can include direct or indirect connections, including connections without direct physical contact. The one or more data store(s) 115 can include sensor data 119. In this context, “sensor data” means any information about the sensor 22, including the capabilities and other information about the sensor.

The communications interface 91 may be configured to enable and/or facilitate communication between the components of the wearable device 26 and entities (such as cloud facilities, cellular and other mobile communications devices, external computing systems, etc.) exterior of the wearable device. For example, the communications interface 91 may enable interaction between a wearer of the wearable device and a cellular device 111 functioning as a screen display to illustrate calibration procedures for the wearable device 26. The communications interface 91 may communicate data over a local-area-network (LAN), a wireless local-area-network (WLAN), a personal-area-network (PAN) (e.g., Bluetooth™), a wide-area-network (WAN), an intranet, the Internet, a peer-to-peer network, point-to-point network, a mesh network, and the like. The communications interface 91 can be a wired and/or wireless network interface.

Referring again to FIG. 1, in one or more arrangements, the sensor 22 may be configured to (via interface 91) interact with a cellular device 111 configured to perform one or more of the functions described herein. For example, in some arrangements, the sensor may be configured to operate in cooperation with a suitable application downloaded onto the cellular device 111 to process sensor data to generate EDA signals and/or other information which may then be relayed back to the wearable device and displayed on the wearable device. In this aspect, the wearable device 26 may utilize the computing power available in the cellular device 111. In other aspects, an external computing system 112 may perform one or more of the functions just described regarding the cellular device 111. In some arrangements, the cellular device 111 may be configured to operate in cooperation with the sensor 22 to function as a display device for displaying (to a user) instructions and/or other information relating to operation of the wearable device 26.

The cellular device 111 and/or the computing system 112 may each also be configured to perform additional functions not mentioned above. The cellular device 111 and the computing system 112 may each communicate with the wearable device 26 via the communications interface 91. For example, wearable device 26 may transmit data indicative of a user's electrodermal activity to one or more external devices (such as cellular device 111 and/or external computing system 112) in example embodiments. When electrodermal activity is detected by EDA sensor circuitry 199, data representative of the electrodermal activity may be communicated, via the communications interface 91 and a suitable communications network, to an external device. The external device may then analyze the data to determine information associated with a user's electrodermal activity. The data and/or one or more control signals may then be utilized to cause the external device to initiate a particular functionality. Generally, the communications interface 91 may be configured to communicate data, such as EDA data, over wired, wireless, or optical networks to and from external devices.

Referring again to FIG. 6, in one or more arrangements, the sensor circuitry may include a power source 55 (such as a battery) operably connected to other elements of the sensor 22 and configured for powering the various operations performed by the electrodes 38, the EDA circuitry 199, and any other active elements of the wearable device. For example, power source 55 may be incorporated into EDA circuitry 199 or coupled to EDA circuitry 199 to provide power for transmission of an electric current through the electrodes 38. The power source 55 may be structured to be removable/replaceable within the sensor 22.

As described herein, by suitable optimization of electrode support structure design parameters and fabrication methods, and in consideration of the pertinent dimensions of an average user's body part both at rest and during exertion, the electrode support structure may be designed and fabricated so that the support structure is always compressed by at least a predetermined amount sufficient to generate at least a predetermined minimum electrode-user contact pressure responsive to contact with the user's skin when the attachment structure is being worn by a user. This contact pressure may be adjusted by varying the tension in the attachment structure or in the contiguous/connected second layers of the support structure securing the wearable device to the user's body part. The predetermined minimum contact pressure may be a contact pressure needed to ensure strong electrical contact between the electrodes and the user's skin. The support structure may be structured so that it is always compressed by at least the predetermined amount when the wearable device is worn by the user, when the user's body part is at rest or inactive.

Detailed embodiments are disclosed herein. However, it is to be understood that the disclosed embodiments are intended only as examples. Therefore, specific structural and functional details disclosed herein are not to be interpreted as limiting, but merely as a basis for the claims and as a representative basis for teaching one skilled in the art to variously employ the aspects herein in virtually any appropriately detailed structure. Further, the terms and phrases used herein are not intended to be limiting but rather to provide an understandable description of possible implementations. Various embodiments are shown in FIGS. 1-6, but the embodiments are not limited to the illustrated structure or application.

While recited characteristics and conditions of the invention have been described in connection with certain embodiments, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

What is claimed is:

1. A dermal sensor comprising:
an electrode support structure including:
at least one multi-material support structure first layer;
at least one multi-material support structure second layer positioned opposite the first layer; and
a plurality of connecting walls extending between and connecting the at least one support structure first layer and the at least one support structure second layer; and
at least a pair of spaced-apart electrodes supported by the support structure.

2. The sensor claim 1, wherein the electrodes are formed integrally with the electrode support structure.

3. The sensor of claim 1, wherein the electrodes are operably connected to the electrode support structure after being formed separately from the electrode support structure.

4. The sensor of claim 1, wherein the electrode support structure is structured to provide a quasi-zero/negative stiffness response over at least a first predetermined range of displacements of one of the support structure first and second layers in a direction toward the other one of the support structure first and second layers.

5. The sensor of claim 4, wherein the electrode support structure is also structured to provide a quasi-zero/negative stiffness response over a second predetermined range of displacements of one of the support structure first and second layers in a direction toward the other one of support structure first and second, the second predetermined range of displacements being different from the first predetermined range of displacements.

6. The sensor of claim 1 wherein the electrode support structure comprises a plurality of support structure unit cells attached end-to-end.

7. The sensor of claim 6 wherein each support structure unit cell includes:
a multi-material unit cell first layer;
a multi-material unit cell second layer positioned opposite the unit cell first layer; and
a plurality of connecting walls extending between and connecting the unit cell first layer and the unit cell second layer.

8. The sensor of claim 7, wherein each multi-material unit cell layer comprises:
at least one flexible section; and
at least one rigid portion attached to the at least one flexible section.

9. The sensor of claim 7, wherein each multi-material unit cell layer comprises a pair of connecting walls extending between and connecting the unit cell first layer and the unit cell second layer.

10. The sensor of claim 1, wherein each connecting wall is formed from a relatively flexible material.

11. The sensor of claim 1, wherein each connecting wall includes:
a first end fixedly attached to the support structure first layer along a flexible section of the first layer; and
a second end opposite first end and fixedly attached to the support structure second layer along a flexible section of the second layer.

12. The sensor of claim 1, further comprising EDA sensor circuitry communicatively coupled to the at least a pair of electrodes and configured to generate an EDA signal responsive to physical contact between the electrodes and a skin surface of a user.

13. The sensor of claim 1, wherein the dermal sensor is an electrodermal activity (EDA) sensor.

14. A wearable device comprising:
an attachment structure structured to be wearable by a user; and
a dermal sensor attached to the attachment structure and including:
an electrode support structure having a multi-material support structure first layer, a multi-material support structure second layer positioned opposite the first layer, and a plurality of connecting walls extending between and connecting the support structure first layer and the support structure second layer; and
at least a pair of spaced-apart electrodes supported by the support structure.

15. The wearable device of claim 14, wherein the wearable device is a wrist band.

16. The wearable device of claim 14, wherein a surface of a first electrode structured to contact a skin surface of a user is structured to be coplanar with a surface of a second electrode structured to contact the skin surface of the user.

17. The wearable device of claim 16, wherein a plane defined by a surface of an electrode support structure separating the first electrode from the second electrode is spaced apart from a plane including the surface of the first electrode and the surface of the second electrode.

18. The wearable device of claim 14, wherein the dermal sensor is an electrodermal activity (EDA) sensor.

19. A dermal sensor comprising:
an electrode support structure including:
a first layer, a second layer positioned opposite the first layer, and a plurality of connecting walls extending between and connecting the support structure first layer and the support structure second layer, the electrode support structure being structured to provide a quasi-zero/negative stiffness response over at least a first predetermined range of displacements of one of the support structure first and second layers in a direction toward the other one of the support structure first and second layers; and
at least a pair of spaced-apart electrodes supported by the support structure.

20. The sensor of claim 19, wherein the dermal sensor is an electrodermal activity (EDA) sensor.
